(19) United States
(12) Patent Application Publication
(10) Pub. No.: US 2015/0290272 A1
(43) Pub. Date: Oct. 15, 2015

(54) PHARMACEUTICAL COMPOSITION FOR PREVENTING/TREATING BRAIN INJURY AND ENHANCING RECOVERY OF SEQUELAE AND MANUFACTURE THEREOF

(71) Applicant: PhytoHealth Corporation, Taipei (TW)

(72) Inventors: Wen-Chi CHEN, Taipei (TW); Hsiao-Chien CHENG, Taipei (TW); Datsen George WEI, Taipei (TW)

(21) Appl. No.: 14/682,863

(22) Filed: Apr. 9, 2015

Related U.S. Application Data
(60) Provisional application No. 61/977,322, filed on Apr. 9, 2014.

(51) Int. Cl. A61K 36/481

(52) U.S. Cl. CPC A61K 36/481

(57) ABSTRACT

The present invention provides a composition for use in the treatment of brain injury and cerebral stoke patients. The composition comprising an effective amount of Astragalus membranaceus extract and pharmaceutically acceptable carrier, excipients or salts. The brain injury includes the traumatic brain injury (TBI) and the acquired brain injury (ABI). The cerebral stoke includes the cerebral ischemia stroke and the cerebral hemorrhage stroke.

### CROSS-REFERENCE TO RELATED APPLICATIONS

This non-provisional application claims priority under 35 U.S.C. §119(a) to Patent Application No. 61/977,322 filed in United States of America on Apr. 9, 2014, the entire contents of which are hereby incorporated by reference.

### FIELD OF THE INVENTION

The present invention relates to a new use of the compositions comprising extracts of Astragalus membranaceus. More particularly, the present inventions of the Astragalus membranaceus can effectively preventing/treating brain injury and enhancing recovery of sequelae.

### BACKGROUND OF THE INVENTION

Brain is the most important part of the human, it is the control center of the activities and emotion. Therefore, the injury of the brain, whether external or internal forces, often bring a significant damage.

Brain injuries occur due to a very wide range of conditions, illnesses, injuries, and as a result of iatrogenesis possible causes of widespread brain damage include birth hypoxia, prolonged hypoxia (shortage of oxygen), poisoning by teratogens (including alcohol), infection, and neurological illness. Chemotherapy can cause brain damage to the neural stem cells and oligodendrocyte cells that produce myelin. Common causes of focal or localized brain damage are physical trauma (traumatic brain injury, stroke, aneurysm, surgery, other neurological disorder), and poisoning from heavy metals including mercury and its compounds of lead.

Brain injury (BI) is the destruction or degeneration of brain cells. Brain injuries occur due to a wide range of internal and external factors. A common category with the greatest number of injuries is traumatic brain injury (TBI) following physical trauma or head injury from an outside source, and the term acquired brain injury (ABI) is used in appropriate circles to differentiate brain injuries occurring after birth from injury due to a disorder or congenital malady.

Stroke, also known as cerebrovascular accident (CVA), cerebrovascular insult (CVI), or brain attack, is when poor blood flow to the brain results in cell death. There are two main types: ischemic due to lack of blood flow (cerebral ischemia) and hemorrhagic due to bleeding (cerebral haemorrhage). About 87% of strokes are ischemic, the rest being hemorrhagic. Some hemorrhages develop inside areas of ischemia, a condition known as “hemorrhagic transformation.” It is unknown how many hemorrhagic strokes actually start as ischemic strokes. They result in part of the brain not functioning properly. Symptoms may include an inability to move or feel on one side of the body, problems understanding or speaking, feeling like the world is spinning, or loss of one vision to one side among others. Symptoms usually but not always come on quickly. If symptoms last less than one or two hours it is known as a transient ischemic attack (TIA). Hemorrhage strokes may also be associated with a severe headache. The symptoms of a stroke can be permanent. Long term complications may include pneumonia or loss of bladder control.

For the Prognosis, stroke can affect people physically, mentally, emotionally, or a combination of the three. The results of stroke vary widely depending on size and location of the lesion. Dysfunctions correspond to areas in the brain that have been damaged.

Some of the physical disabilities that can result from stroke include muscle weakness, numbness, pressure sores, pneumonia, incontinence, apraxia (inability to perform learned movements), and difficulties carrying out daily activities, appetite loss, speech loss, vision loss and pain. If the stroke is severe enough, or in a certain location such as parts of the brainstem, coma or death can result.

A cerebral haemorrhage is a subtype of intracranial hemorrhage that occurs within the brain tissue itself. It is alternatively called intracerebral hemorrhage (ICH). It can be caused by brain trauma, or it can occur spontaneously in hemorrhagic stroke. Non-traumatic intracerebral hemorrhage is a spontaneous bleeding into the brain tissue. Non-traumatic can refer to increased exception, tension or stress. ICH accounts for 10-30% of all stroke admissions to hospital, and leads to catastrophic disability, morbidity, and a 6 month mortality of 30-50%. Long-term outcomes are poor; only 20% of patients regain functional independence at 6 months. Although there have been therapeutic advances for aneurysmal subarachnoid haemorrhage and cerebral infarction, treatment for ICH remains limited. Depending on the underlying cause of haemorrhage, ICH is classified as primary or secondary. Primary ICH is when the haemorrhage originates from spontaneous rupture of small arteries or arterioles damaged by chronic hypertension or amyloid angiopathy. Secondary ICH is when haemorrhage results from trauma, rupture of an aneurysm, vascular malformation, coagulopathy, or other causes.

Cerebral ischemia is a condition in which there is insufficient blood flow to the brain to meet metabolic demand. This leads to poor oxygen supply or cerebral hypoxia and thus to the death of brain tissue or cerebral infarction/ischemic stroke. It is a sub-type of stroke along with subarachnoid hemorrhage and intracerebral hemorrhage. Ischemia leads to alterations in brain metabolism, reduction in metabolic rates, and energy crisis. There are two types of ischemia: focal ischemia, which is confined to a specific region of the brain; and global ischemia, which encompasses wide areas of brain tissue.

In an ischemic stroke, blood supply to part of the brain is decreased, leading to dysfunction of the brain tissue in that area. There are four reasons why this might happen: (1) Thrombosis (obstruction of a blood vessel by a blood clot forming locally). (2) Embolism (obstruction due to an embolus from elsewhere in the body, see below). (3) Systemic hypoperfusion (general decrease in blood supply, e.g., in shock). (4) Venous thrombosis.

Currently, the thrombolysis agent recombinant tissue plasminogen activator (rt-PA) (Actilyse) is a unique and effectively drug for the acute ischemic stroke. The rtPA, in acute ischemic stroke, when given within three hours of symptom onset results in an overall benefit of 10% with respect to living without disability. It does not, however, improve chances of survival. Benefit is greater the earlier it is used. Between three and four and a half hours the effects are less clear. A 2014 review found a 5% increase in the number of people living without disability at three to six months; however, there was a 2% increased risk of death in the short term. After four and a half hours thrombolysis worsens outcomes. These benefits or lack of benefits occurred regardless of the age of the person treated. There is no reliable way to determine who will have an intracranial hemorrhage post treatment versus who will not.

Its use is endorsed by the American Heart Association and the American Academy of Neurology as the recommended treatment for acute stroke within three hours of onset of symptoms as long as there are not other contraindications (such as abnormal lab values, high blood pressure, or recent surgery). This position for rt-PA is based upon the findings of two studies by one group of investigators which showed that rt-PA improves the chances for a good neurological outcome. When administered within the first three hours thrombolysis improves functional outcome without affecting mortality. 6.4% of people with large strokes developed substantial brain hemorrhage as a complication from being given tPA thus part of the reason for increased short term mortality. Additionally, it is the position of the American Academy of Emergency Medicine that objective evidence regarding the efficacy, safety, and applicability of rt-PA for acute ischemic stroke is insufficient to warrant its classification as standard of care.

Although the preview literature has discourse the Chinese herbal formula containing Astragalus can therapeutic efficacy of stroke. The Chinese herbal formula such as: buyanghuanwu soup (including 120 grams of Astragalus, 6 grams of Angelica tail, 5 grams of red peony, 3 grams of earthworm, 3 grams of Chuanxiong, 3 grams of walnuts and 3 grams of saffron). However, there are no prior literature has discourse the single drug or a Chinese herbal extracts can be effective in treating stroke.

According to the principle of the Chinese herb, the therapeutic effect is quite different from the single medicinal material to the mixture medicinal material. Therefore the mixture Chinese herbal containing Astragalus cannot directly explain that the Astragalus extract also has the therapeutic effect to the stroke.

Therefore, the brain injury and the cerebral stoke patients, in clinical, need an effect therapy drug for more safety, lower side effect, and no use time limit.

### SUMMARY OF THE INVENTION

The present invention provides a composition of the Astragalus membranaceus extract comprise the 60%-120% totally polysaccharides. The polysaccharides characteristic are obtained by the Fourier transform infrared spectroscopy (FTIR) and the Nuclear Magnetic Resonance Spectroscopy (NMR). The polysaccharides bond linkage include α-1,4 glycosidic bond linkage and α-1,6 glycosidic bond linkage. The main bond linkage site of the arabinose is at the carbon terminal, C3 and C5. The main bond linkage site of the bond linkage of the glycan is at the carbon terminal, C4 and C6. The main bond linkage site of the galactose is at the carbon terminal, C3 and C6. The main bond linkage site of the galacturonic acid is at the C4. The main bond linkage site of the rhamnose is at the C2. The compositions further comprise a first active agent.

The present invention provides a composition for use in the prevention and treatment of brain injury comprising an effective amount of Astragalus membranaceus extract and pharmaceutically acceptable carrier, excipients or salts. The brain injuries include the traumatic brain injury (TBI) and the acquired brain injury (ABI). The Astragalus membranaceus extract uses for the preventing, cure and prognosis of the brain injury patients. In some embodiments, the Astragalus membranaceus extract concentration is between 125 mg˜2000 mg/day.

In some embodiments, the composition of the Astragalus membranaceus extract prevention and treatment of brain injury comprise: 60%-120% totally polysaccharides; α-1,4 glycosidic bond linkage; α-1,6 glycosidic bond linkage; bond linkage of arabinose at terminal carbon, C3 and C5; bond linkage of glycan at terminal carbon, C4 and C6; bond linkage of galactose at terminal carbon, C3 and C6; bond linkage of galacturonic acid at C4; and bond linkage of rhamnose at C2.

The present invention provides a composition for use in the prevention and treatment of the stoke comprising an effective amount of Astragalus membranaceus extract and pharmaceutically acceptable carrier, excipients or salts. The cerebral stoke include the cerebral ischemia stroke and the cerebral haemorrhage stroke. The Astragalus membranaceus extract use for the preventing, cure and prognosis of the stoke patients. In some embodiments, the effective amount of Astragalus membranaceus extract is between 125 mg˜2000 mg/day.

The present invention provides a method for preparing extract of Astragalus membranaceus comprising:
1) wash and clean the dry of Astragalus membranaceus to get a clean Astragalus membranaceus and further mechanically processed into drink chips or fine powder.
2) put the processed Astragalus membranaceus drink chips or fine powder into an extract solution to extract at temperature 80° C. to 100° C., for 2 hrs to 3 hrs to get the a first extract solution, then repeat 2 times at same temperature, for 1.5 hrs to 3 hrs.
3) concentrate all of the extract solutions, then use ethanol or lower alkanol to be precipitated and separated at low temperatures; and
4) put the selected precipitates though centrifugation ultra filter and/or ion exchanger, following high molecular weight cut-off membrane.

The details of one or more embodiments of the invention are set forth in the description below. Other features or advantages of the present invention will be apparent from the following detailed description of an example and also from the appended claims.

### BRIEF DESCRIPTION OF THE DRAWINGS

FIG. 1 illustrates the NMR analysis of the Astragalus membranaceus extract.

FIG. 2. ICH patients who received Astragalus membranaceus extract were highly improved the degree of recovery compared with control group with significantly difference in the 84th day GOS score after stroke.

FIG. 3. ICH patients who received Astragalus membranaceus extract were highly improved the degree of recovery compared with control group with significantly difference in the 84th day mRS score after stroke.

FIG. 4. LDH activity under OGD-induced neurotoxic conditions were measured in PCC with Astragalus membranaceus extract treatment of various dosage

FIG. 5A˜FIG. 5B. illustrates that Astragalus membranaceus extract facilitates the stroke recovery by reducing the infarct volume.

FIG. 5C. illustrates that the infarct volume as assessed by magnetic resonance imaging was significantly reduced in Astragalus membranaceus extract-treated rats (50 mg/kg) as compared with vehicle control.

FIG. 5D˜FIG. 5E. illustrates the quantification of infraction volume and area of the largest infarcted slice with the 50 mg dosage the Astragalus membranaceus extract treatment in the cerebral ischemia rat.

FIG. 6A. Illustrates the Ischemic rats receiving Astragalus membranaceus extract showed significantly reduced body asymmetry compared with control rats.

FIG. 6B˜FIG. 6D. Locomotor activities such as vertical activity, vertical movement time, and number of movements significantly increased in rats receiving Astragalus membranaceus extract treatment compared with control rat.

FIG. 6E. illustrates that a higher ratio of improvement in grip strength was founded in Astragalus membranaceus extract treatment group compared with control group

### DETAILED DESCRIPTION OF THE INVENTION

### Embodiment 1

Manufacture the Astragalus membranaceus Extract

This teaching describes how an extract is isolated from Astragalus membranaceus and purified. In some embodiments, the roots of two-year old plants of Astragalus membranaceus provide a good source of the composition. In many embodiments, the roots can be from, for example, A. membranaceus Bge. var. mongholicus (Bge.) Hsiao; A. membranaceus (Fisch.) Bge.; or, A. membranaceus grown in Inner Mongolia or Shanxi province, Peoples' Republic of China.

The extract from the Astragalus membranaceus is typically derived from sterile processed chipped or sectioned dried roots. The preparation includes trimming the dried roots, scrubbing them with ultrafiltered (UF) water, and cleaning them with a disinfecting solution, such as 70% to 75% ethanol. The roots are cut into thin slices and dried under sterile conditions to produce “drink chips,” also referred to herein as “clean chips.”

The extract is comprised of the polysaccharides that are isolated from the drink chips or fine powders using a process that includes applying an aqueous extraction solvent having a temperature of at least 80-100° C.

The extraction solvent can be water and can optionally include a co-extractant, such as an alkaline earth metal salt or an alkali metal salt. The alkaline earth metal salt or alkali metal salt can include, for example, calcium oxide, potassium dihydrogen phosphate or sodium dihydrogen phosphate. The co-extractant can be, for example, at a concentration of 0.5M KH₂PO₄ at a pH of about 4.5).

The extraction solvent is applied to the drink chips or fine powders for a time, temperature, and for as many extraction cycles needed to isolate the extract from the drink chips or fine powders. A typical process includes three extraction cycles, each cycle lasting about1.5 hours to 3 hours and applying an extraction solvent having a temperature of about 80-100° C. in some embodiments, the drink chips are extracted with an initial hot aqueous salt wash, such as in 0.5 M KH₂PO₄ at a pH of 4.5 and at a temperature of about 90-100° C. for about 30 minutes. In some embodiments, all steps following the preparation of the drink chips or fine powders are conducted under aseptic conditions that include the use of sterile equipment and reagents.

The extraction solvent is then concentrated, and this can be done under vacuum at a temperature ranging from about 60° C. to about 70° C., to achieve a concentration of about 0.8-1.1 L per 1 kg of the drink chips or fine powders. The concentration can also be accomplished using ultrafiltration, such as ultrafiltration with a 100 kiloDalton molecular weight cut-off filter.

The extract is then precipitated from the concentrated extraction solvent using a lower alkanol. The lower alkanol can be, for example, ethanol. In some embodiments, the ethanol can be added to the solvent to a concentration of about 70% ethanol at about room temperature to create the precipitate. In some embodiments, the precipitation is done by first using a lower concentration of about 35-40% ethanol in a first precipitation step, and then using a higher concentration of about 65-80% ethanol in a second precipitation step. The concentrations of lower alkanol used in the precipitations can range from, for example, 20%-30%, 30%-40%, 40%-50%, 50%-60%, 60%-70%, 70%-80%, 80%-90%, or any concentration therein.

The alkanol washes can be repeated for further purification. The precipitate, for example, can be washed with more of the lower alkanol, where a typical wash may include, for example, three or four washes with 95% ethanol or 99%ethanol. The precipitate is then suspended in water at a concentration suitable for further processing such as, for example, about 18-20% weight/volume. Reprecipitation by again adding a lower alkanol in the water can be used to remove additional materials that are not water-soluble. The supernatant can be precipitated with a higher concentration of the lower alkanol. The higher concentration of the lower alkanol can be, for example, 40-80% ethanol, and in some embodiments 60-70% ethanol, to create a crude extract precipitate that contains an arabinogalactan protein and associated polysaccharides.

The crude extract is re-dissolved in water and dried. Suitable drying processes can include any process known to one of skill that avoids excessive heating and can include, for example, spray drying, vacuum drying, freeze-drying, critical point drying, solvent exchange, and the like. The crude extract can also be re-di ssolved in water or aqueous solution, wherein the crude extract is brought to a suitable concentration for ultrafiltration. In some embodiments, the suitable concentration for ultrafiltration is about 2-5%. The crude extract can then be ultrafiltered to further remove low molecular weight materials and reduce the volume of the solution, wherein the ultrafiltration system, for example, can have a 5-6 kiloDalton molecular weight cut-off.

In some embodiments, the crude extract can appear as a white to off-white powder, or light yellow powder, and is soluble in water to at least about 100 mg/mL. In some embodiments, the powder is soluble in water to at least about 200 mg/mL. The powder shows a weight loss upon drying of less than about 15%, has an endotoxin content of less than about 0.5 EU/mg and, in some embodiments, has an endotoxin content of less than about 0.3 EU/mg.

The crude extract can be further purified by macroreticular chromatography or ion-exchange chromatography. The composition is re-dissolved in an aqueous solution and brought to a suitable concentration for ultrafiltration, typically a concentration of about 0.5-2%. The redissolved composition is then ultrafiltered to further remove low molecular weight materials and reduce the volume of the solution. The 5 kiloDalton molecular weight cut-off ultrafiltration system described above can be used. The retentate of the ultrafiltration is eluted through a cation exchange column, such as a SP Sepharose cation exchange column equilibrated with 20 mM NaOAc buffer at pH 5.20. The eluate from the cation exchange column is eluted through an anion exchange column, such as a Q Sepharose anion exchange column equilibrated with the same NaOAc buffer.

The eluate from the anion exchange column may be (1) used directly in the preparation of other forms of the extract as taught herein; (2) concentrated and dried to form the crude extract, which can be kept as an intermediate suitable for preparation of the purified extract; or (3) used directly in the preparation of the purified extract.

In the preparation of the purified extract, the crude extract may be microfiltered through a suitable bacteriostatic filter, such as a 0.1 μm filter, and ultrafiltered to desalt the solution and again reduce its volume. An 8 kiloDalton molecular weight cut-off ultrafiltration may be used, in some embodiments. The retentate from the ultrafiltration is concentrated, and the percent concentration can be determined using a refractometer. In some embodiments, the retentate is concentrated to a concentration of about 20-26% at 50-60° C. The concentrated retentate is then precipitated with a lower alkanol. In some embodiments, the lower alkanol can be ethanol that is added to a concentration of about 80-90%. The precipitate may be further washed, where a typical wash can include three washes of the precipitate with anhydrous ethanol. The precipitate is then dried to give the purified extract. In some embodiments, the drying of the precipitate can occur, for example, through use of a vacuum oven or a spray drying method to dry the precipitates at a temperature of about 60-70° C.

### Embodiment 2

The Purified Extract Determined by NMR

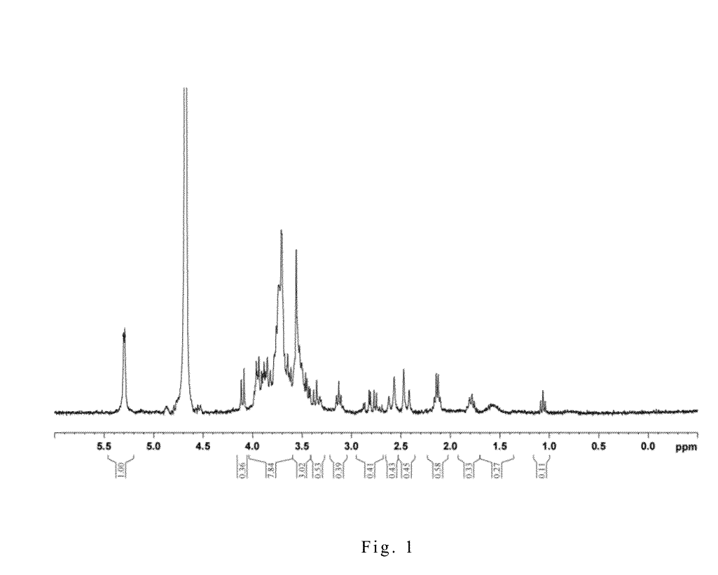

The composition of the purified extract can be determined by NMR. ¹H NMR spectra is recorded at a probe temperature on a spectrometer. Samples were exchanged twice with 99.9 atom % D₂O with intermediate lyophilization, finally dissolved in D₂O. 1H chemical shifts are expressed in ppm by reference to internal acetone. 1D 300-MHz 1H NMR spectra were recorded. Methylation analysis is also used for glycosidic linkages. In this procedure the molecule can be first completely O-methylated. The products can be converted by successive hydrolysis to give partially O-methylated units, which are reduced with NaBH4, followed by acetylation to provide partially O-methylated units, which on GC-MS, have typical retention times and electron impact spectra. The polysaccharide extracted from sequential purification steps consisting of L-rhamnose, L-arabinose, D-glucose, and D-galactose with main chain for 1,6-and 1,2,6-galactopyranosyl and 1,5-and 1,3,5-arabinofuranosyl residues, and minor chain for 1,4-and 1,4,6-glucopyranosyl and 1,2-and 1,2,4-rhamnofuranosyl residues exhibits biological functions associated with following in vivo/in vitro neuronprotection. (FIG. 1)

It is clear that the Astragalus membranaceus extract is a heterogeneous structure of polysaccharides with complex conformation. With systematic analysis of the Astragalus membranaceus extract, it can be concluded that there are two types of polysaccharides in the sample: one α-1,4-linkage form and one α-1,6-linkage form. The relative composition of these type of carbohydrate moieties (α-1,4: α-1,6) were determined as 4:1˜8:1 by ¹H NMR spectra.

### Embodiment 3

Clinical Interpretation and Use of Stroke Scales

1. Glasgow Outcome Scale (GOS): Glasgow Outcome Scale was developed to define broad outcome categories for people who sustain acute brain damage from head injury or non-traumatic brain insults. The scale reflects disability and handicap rather than impairment; that is, it focuses on how the injury has affected functioning in major areas of life rather than on the particular deficits and symptoms caused by injury. It is not intended to provide detailed information about the specific difficulties faced by individual patients, but to give a general index of overall outcome. The GOS is a one item scale with 5 possible ratings (Dead, Vegetative State, Severe Disability, Moderate Disability, and Good Recovery).

2. The modified Rankin Scale (mRS): The modified Rankin Scale (mRS) is a commonly used scale for measuring the degree of disability or dependence in the daily activities of people who have suffered a stroke or other causes of neurological disability. It has become the most widely used clinical outcome measure for stroke clinical trials.

3. Barthel Index (BI): The Barthel Index is an ordinal scale used to measure performance in activities of daily living (ADL). Each performance item is rated on this scale with a given number of points assigned to each level or ranking. It uses ten variables describing ADL and mobility. A higher number is associated with a greater likelihood of being able to live at home with a degree of independence following discharge from hospital. The amount of time and physical assistance required to perform each item are used in determining the assigned value of each item.

4. Functional independence measure (FIM): The Functional Independence Measure (FIM) is an assessment tool that aims to evaluate the functional status of patients throughout the rehabilitation process following a stroke, traumatic brain injury, spinal cord injury or cancer. Its area of use can include skilled nursing facilities and hospitals aimed at acute, sub-acute and rehabilitation care. It serves as a consistent data collection tool for the comparison of rehabilitation outcomes across the health care continuum.

### Embodiment 4

Favorable Clinical Outcome of sICH in Patients Treated with Astragalus membranaceus Extract

1. Study Design and Intervention

The study enrolled male and female patients, average aged 42-70 with acute hemorrhagic stroke based on a clinical diagnosis. A total of 47 patients (experiment group comprehends 22 patients and control group comprehends 25 patients).

Each group was treated as follows: 1) control group accepted placebo (saline solution); 2) experiment group accepted Astragalus membranaceus extract t.i.w treatment for 14 days from second day of admission in addition to standard ordinary treatment.

The experiment group use the Astragalus membranaceus extract composition of this invention (comprehends 22 patients). The control group use the saline solution (comprehends 25 patients).

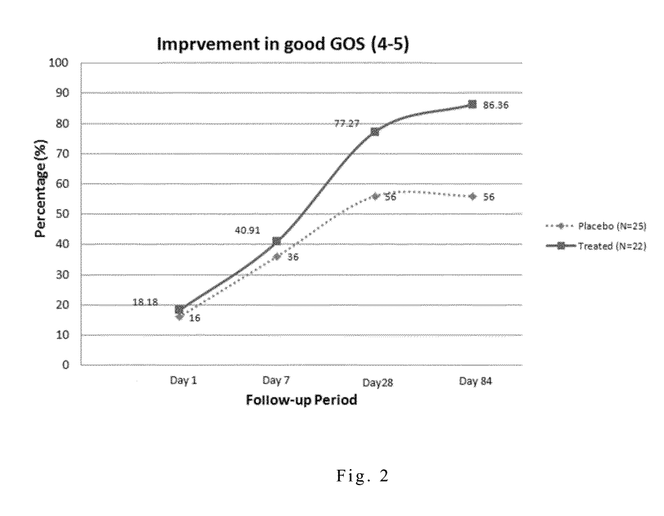

Comparing the patients' GOS scores (4-5) proportion of the experiment group and the control group. (4-5: moderate disability to good recovery (low sequelae); 0-3: death to severe disability). Within three months after stroke, no matter during the treatment period (seven days after stroke) or after treatment (28 and 84 days after stroke) were observed. The result shows the proportion of patients with good GOS scores (4-5) in the experiment group is highly than that in the control group. (FIG. 2) The observation of the 84 days after stroke shows the difference was more than 30 percent. During the time increase, the proportions of the magnitude are more obvious than the placebo treatment group. These results indicate the Astragalus membranaceus extract of this invention can cure the sequela of the cerebral stroke and increase the patients recover (FIG. 2). Functional outcome measures in the 84th days, the GOS in 4-5 of placebo group was 56%, and of the experiment group was 86.4%.

2.The Modified Rankin Scale (mRS) Test

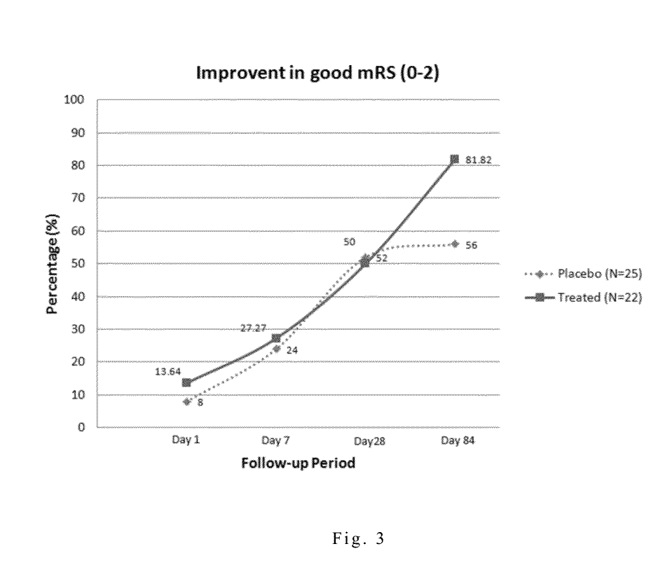

Comparing the patients' mRS scores (0-2) proportion of the experiment group and the control group. (0-2: no disability to mild disability (Low sequelae); 3-5: moderate to severe disability). The result shows the experiment group mRS scores (0-2) proportion are highly than the control group. (FIG. 3) The observation of the 84 days after the stroke shows the difference was more than 25 percent. These results indicate the Astragalus membranaceus extract of this invention can cure the sequela of the cerebral stroke and increase the patients recover (FIG. 3). In the 84th days, the mRS in 0-2 of placebo group was 56%, and of the treatment group was 81.8%.

3. The TNF Measurement

The experiment group use the Astragalus membranaceus extract composition of this invention (comprehends 22 patients). The control group uses the saline solution (comprehends 25 patients). The TNF levels of treatment group were significantly lower than placebo group in the 7th day 14th days compared to the 1st day of ICH occurred, Since TNFα is one of the key players in stroke progression, in the present study, the result indicates the Astragalus membranaceus extract of this invention can cure the sequela of the cerebral stroke and increase the patients recovery (Table 1).

TABLE 1
TNF measurement

| | Placebo | Treatment | | |
|---|---|---|---|---|
| Variable | (N = 25) | (N = 22) | P value^{&} | P value [symbol] |
| TNF | | | | |
| DAY1 | 10.23 ± 27.35 | 15.35 ± 47.43 | 0.66 | 0.69 |
| DAY4 | 11.13 ± 29.71 | 13.25 ± 37.68 | 0.83 | 0.60 |
| DAY7 | 25.71 ± 62.15 | 8.81 ± 22.26 | 0.21 | 0.29 |
| DAY14 | 23.58 ± 55.45 | 4.93 ± 6.78 | 0.11 | 0.60 |
| DAY4 − DAY1 | 0.90 ± 3.27 | −2.10 ± 11.61 | 0.25 | 0.29 |
| DAY7 − DAY1 | 15.48 ± 54.34 | −6.54 ± 26.28 | 0.08 | 0.01 |
| DAY14 − DAY1 | 13.35 ± 42.03 | −10.42 ± 42.01 | 0.06 | 0.10 |
| (DAY4 − DAY1)/DAY1 | 0.44 ± 1.32 | −0.18 ± 0.49 | 0.04 | 0.14 |
| (DAY7 − DAY1)/DAY1 | 1.86 ± 3.41 | 0.02 ± 1.05 | 0.02 | 0.03 |
| (DAY14 − DAY1)/DAY1 | 1.43 ± 3.91 | 1.64 ± 7.39 | 0.91 | 0.15 |

Data presented as mean ± standard deviation.
*p < 0.05;
**p < 0.01;
***p < 0.001 for paired test;
^{&}independent test for continuous data;
$: Wilcoxon rank sum test

4. The Relationship of the TNF and the Stroke Prognosis Index (GOS and mRS)

According to the experiment result, the analysis of TNF level and GOS scores were inversely correlated, but TNF level and mRS scores were correlated directly (Table 2). It has been known that plasma TNF is elevated in patients with acute stroke. These results indicate that the Astragalus membranaceus extract of this invention can cure the sequela of the cerebral stroke and increase the patients recovery.

TABLE 2
The relationship of the TNF and the cerebral stroke prognosis index (GOS, mRS)

| | TNF | | | | | | | | | |
|---|---|---|---|---|---|---|---|---|---|---|
| | DAY1 | DAY4 | DAY7 | DAY14 | D1 | D2 | D3 | PI | P2 | P3 |
| GOS | | | | | | | | | | |
| DAY1 | −0.10 | −0.11 | −0.05 | −0.12 | 0.04 | 0.03 | −0.02 | 0.11 | 0.00 | −0.08 |
| DAY7 | 0.14 | 0.11 | −0.01 | −0.17 | −0.22 | −0.13 | −0.29* | 0.27 | 0.11 | −0.19 |
| DAY28 | 0.00 | −0.05 | −0.16 | −0.33* | −0.20 | −0.17 | −0.31* | 0.01 | 0.07 | −0.01 |
| DAY84 | −0.01 | −0.04 | 0.11 | −0.15 | −0.10 | 0.13 | −0.13 | 0.04 | 0.21 | −0.04 |
| DAY7 − DAY1 | 0.30* | 0.25 | 0.04 | −0.11 | −0.33* | −0.21 | −0.36* | 0.24 | 0.14 | −0.16 |
| DAY28 − DAY1 | 0.08 | 0.03 | −0.12 | −0.24 | −0.24 | −0.20 | −0.30* | −0.07 | 0.07 | 0.05 |
| DAY84 − DAY1 | 0.06 | 0.04 | 0.14 | −0.06 | −0.12 | 0.10 | −0.11 | −0.03 | 0.21 | 0.01 |
| (DAY7 − DAY1)/DAY1 | 0.30* | 0.26 | 0.04 | −0.11 | −0.33* | −0.21 | −0.37* | 0.24 | 0.14 | −0.16 |
| (DAY28 − DAY1)/DAY1 | 0.08 | 0.03 | −0.12 | −0.24 | −0.24 | −0.20 | −0.30* | −0.07 | 0.07 | 0.06 |
| (DAY84 − DAY1)/DAY1 | 0.06 | 0.04 | 0.13 | −0.06 | −0.12 | 0.09 | −0.11 | −0.03 | 0.18 | 0.01 |
| MRS | | | | | | | | | | |
| DAY1 | 0.05 | 0.04 | 0.06 | 0.12 | −0.04 | 0.02 | 0.07 | −0.14 | 0.07 | 0.12 |
| DAY7 | −0.06 | −0.05 | 0.02 | 0.19 | 0.10 | 0.07 | 0.24 | −0.29 | −0.15 | 0.18 |
| DAY28 | 0.02 | 0.06 | 0.12 | 0.29* | 0.13 | 0.11 | 0.26 | −0.03 | −0.15 | −0.06 |
| DAY84 | −0.08 | −0.06 | −0.18 | 0.08 | 0.12 | −0.13 | 0.14 | 0.01 | −0.21 | 0.02 |
| DAY7 − DAY1 | −0.17 | −0.13 | −0.04 | 0.16 | 0.23 | 0.10 | 0.30* | −0.30 | −0.31* | 0.13 |
| DAY28 − DAY1 | −0.01 | 0.03 | 0.10 | 0.25 | 0.19 | 0.12 | 0.25 | 0.07 | −0.22 | −0.15 |
| DAY84 − DAY1 | −0.13 | −0.10 | −0.25 | −0.01 | 0.17 | −0.16 | 0.10 | 0.12 | −0.29 | −0.07 |
| (DAY7 − DAY1)/DAY1 | −0.10 | −0.07 | 0.00 | 0.16 | 0.18 | 0.09 | 0.24 | −0.23 | −0.25 | 0.15 |
| (DAY28 − DAY1)/DAY1 | 0.02 | 0.06 | 0.12 | 0.27 | 0.17 | 0.12 | 0.24 | 0.08 | −0.20 | −0.11 |
| (DAY84 − DAY1)/DAY1 | −0.09 | −0.06 | −0.21 | 0.03 | 0.15 | −0.15 | 0.11 | 0.13 | −0.27 | −0.03 |

GOS: Glasgow outcome scale;
MRS: Modified Rankin scale;
*P<0.05
D1 = DAY4 − DAY1; D2 = DAY7 − DAY1; D3 = DAY14 − DAY1
P1 = (DAY4 − DAY1)/DAY1; P2 = (DAY7 − DAY1)/DAY1; P3 = (DAY14 − DAY1)/DAY1

### Embodiment 5

Astragalus membranaceus Extract Treatment of Cerebral Schemia Stroke in vitro Model

In vitro PCC preparation and oxygen glucose deprivation treatment. Primary cortical cells (PCC) were prepared from the cerebral cortex of gestation day 17 embryos from Sprague-Dawley rats as described previously with modification. Four days after isolation, the cultures were replenished with minimum essential medium (MEM, GIBCO-BRL) containing 0.5 g/L BSA and 2% B27 supplement, 0.5 mM pyruvate and antibiotics. Finally, the culture medium was changed to serum-free neurobasal medium containing 1 mM pyruvate, 1 mM glutamate, 0.5 g/L BSA, 2% B27 supplement, and antibiotics on the seventh day. For oxygen glucose deprivation (OGD) treatment, the cells cultured with glucose-free Earle's balanced salt solution were placed for 6-8 hours within a hypoxic chamber (Bug Box, Ruskinn Technology), continuously flushed with 95% N2 and 5% CO2 at 37° C. to maintain a gas phase PO2 of <1 mmHg (OM-14 oxygen monitor; SensorMedics Corporation). Control cells were incubated in glucose-free Earle's balanced salt solution in a normoxic incubator for the same period. OGD was terminated by switching back to normal culture conditions.

LDH Assay Method

In order to prove the concept of neuroprotection of the Astragalus membranaceus extract, PCC were prepared in 24-well plates and pre-treated with various concentration of the Astragalus membranaceus extract (0.1, 1, and 10 μM). After 20 minutes of the Astragalus membranaceus extract pretreatment, PCC were subjected to OGD in the hypoxia chamber for 6-8 hours, and then the culture media were collected for lactate dehydrogenase (LDH) activity assays as described (J Neurosci Methods 1987; 20:83-90).

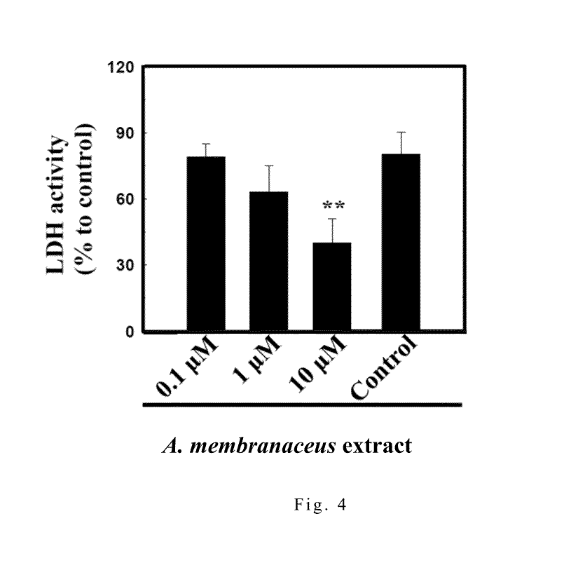

To evaluate the neuroprotectivity of the Astragalus membranaceus extract in vitro, LDH activity under OGD-induced neurotoxic conditions were measured in PCC with the Astragalus membranaceus extract treatment of various dosage (0.1, 1, and 10 μM) (FIG. 4). Treatment with 10 μM the Astragalus membranaceus extract before OGD significantly reduced LDH activity in comparison with the control (FIG. 4).

Astragalus membranaceus Extract Treatment of Cerebral Ischemia Stroke in vivo Ischemia Animal Model

In vivo ischemia animal model. Adult male Sprague-Dawley rats (weight 250-300 g) were used for this study. The rats were anesthetized with chloral hydrate (0.4 g/kg, ip) and subjected to right middle cerebral artery (MCA) ligation and bilateral common carotid artery (CCA) clamping as previously described (Stroke 1986; 17:738-743). Briefly, the bilateral CCAs were clamped with non-traumatic arterial clips. Using a surgical microscope, a 2×2 mm craniotomy was drilled at the point where the zygoma fuses to the squamosal bone, the right MCA was then ligated with a 10-0nylon suture. Cortical blood flow was measured continuously with a laser Doppler flowmeter (PF-5010, Periflux system, Perimed AB, Stockholm, Sweden) in anesthetized animals. A photodetector probe (0.45 mm in diameter) was stereotaxically placed through a skull burr hole (1 mm in diameter) in the frontoparietal cortex (1.3 mm posterior, 2.8 mm lateral to the bregma, and 1.0 mm below the dura). Then, experimental rats were injected intravenously with the Astragalus membranaceus extract (1, 10, 25, and 50 mg/kg in saline) or vehicle 30 minutes after MCA ligation for three consecutive days through a 26-gauge syringe into the right femoral vein. After 90 minutes of ischemia, the 10-0 suture on the MCA and arterial clips on CCAs were removed to allow for reperfusion. During recovery from anesthesia, body temperature was maintained at 37° C. with a heat lamp.

1. Neurological Behavioral Measurements.

Behavioral assessments were performed 3 days before cerebral ischemia, and 72 hours after cerebral ischemia. The tests measured body asymmetry and locomotor activity as previously described (Stroke 2003; 34:558-564). Further, grip strength was analyzed using Grip Strength Meter (TSE-Systems) as previously described with modification (Neurosci Lett 1998; 246:1-4). In brief, percentage of improvement in grip strength was measured on each forelimb separately and was calculated as the ratio between the mean strength out of 20 pulls of the side contralateral to the ischemia and the ip silateral side. In addition, the ratio of grip strength post-treatment and baseline were also calculated and changes were presented as a percentage of baseline value.)

2. Triphenyltetrazolium Chloride (TTC) Staining

Three days after cerebral ischemia, animals were intracardially perfused with saline. The brain tissue was removed, immersed in cold saline for 5 min, and sliced into 2.0-mm-thick sections (seven slices per rat). The brain slices were incubated in 20 g/L TTC (Research Organics Inc), dissolved in saline for 30 min at 37° C., and then transferred to a 5% formaldehyde solution for fixation. The area of infarction in each slice was measured with a digital scanner, as described previously (J Neurosci 1997; 17:4341-4348). The volume of infarction was obtained from the product of average slice thickness (2 mm) and by examining infarcted areas in all brain slices. To minimize any artifacts induced by post-ischemic edema in the infarcted tissue, the area of infarction was also calculated as previously described (Stroke 1993; 24:117-121). To measure the infarcted area in the right cortex, we subtracted the non-infarcted area in the right cortex from the total cortical area of the left hemisphere.

3. Measurement of the Infarct Size Using Magnetic Resonance Image (MRI)

MRI was performed on rats under anesthesia in a General Electric imaging system (GE, R4) at 3.0 T. Brains were scanned in 6 to 8 coronal image slices, each 2 mm thick without any gaps. T2-weighted imaging (T2WI) pulse sequences were obtained with the use of a spin-echo technique (repetition time, 4000 ms; echo time, 105 ms) and were captured for each animal at 14 days after cerebral ischemia. To measure the infarction area in the right cortex, we subtracted the noninfarcted area in the right cortex from the total cortical area of the left hemisphere. The area of infarct was drawn manually from slice to slice, and the volume was then calculated by internal volume analysis software (Voxtool, General Electric).

Refer to all of the embodiments in the specification, all results indicate the effect of Astragalus membranaceus extract with modified manufacture is greater than the effect of Astragalus membranaceus extract with ordinary manufacture.

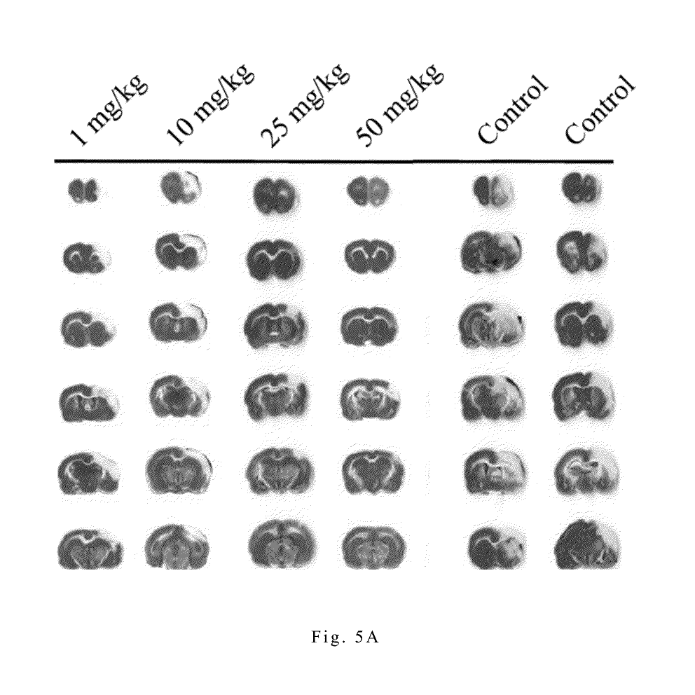

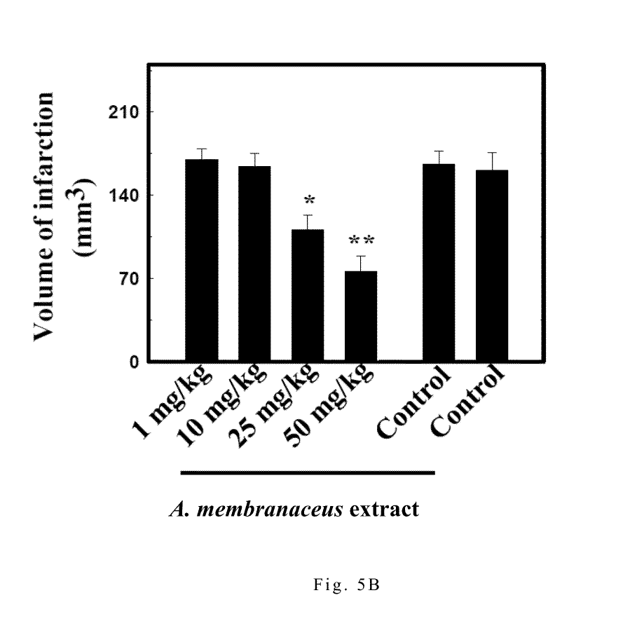

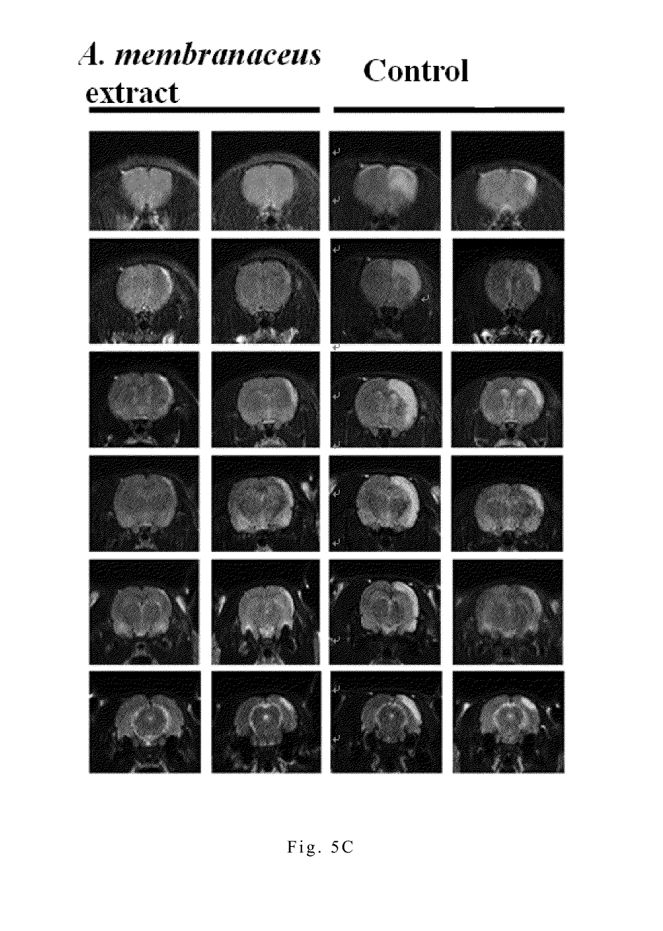

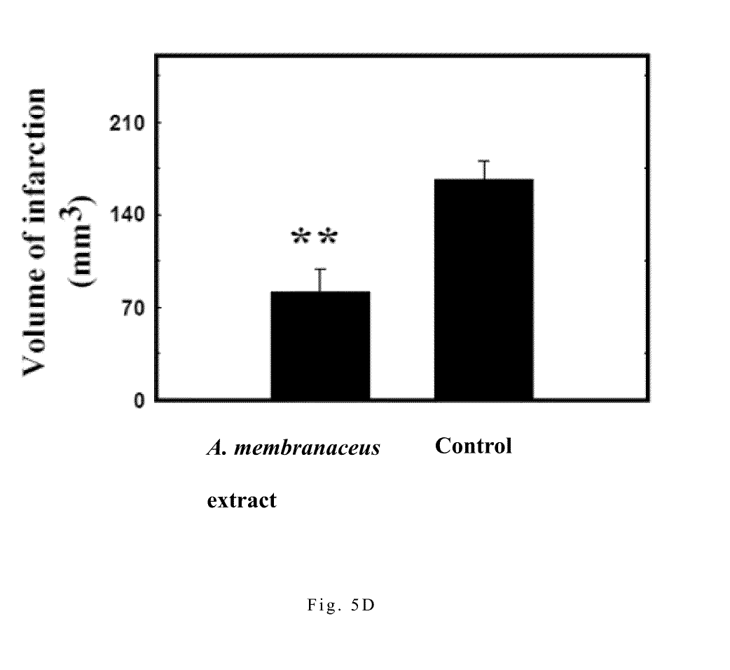

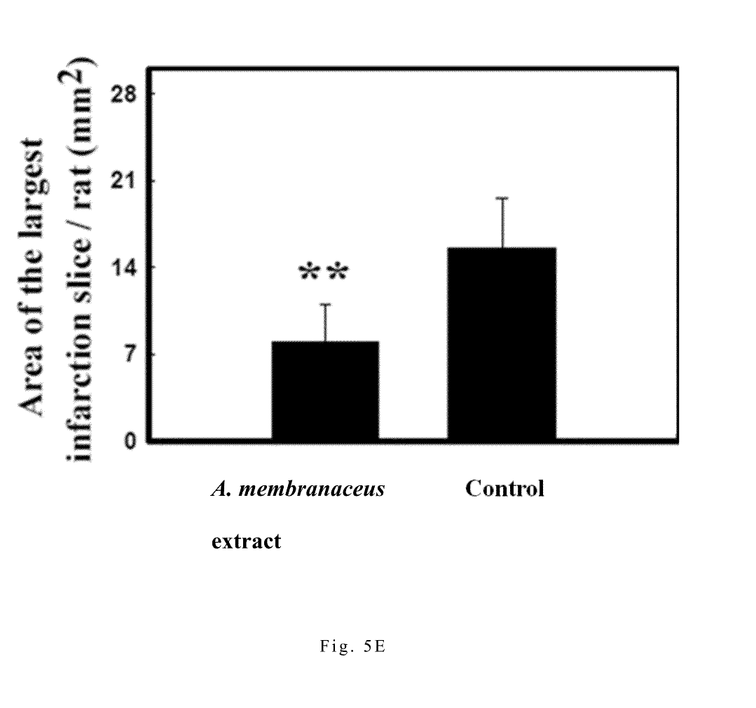

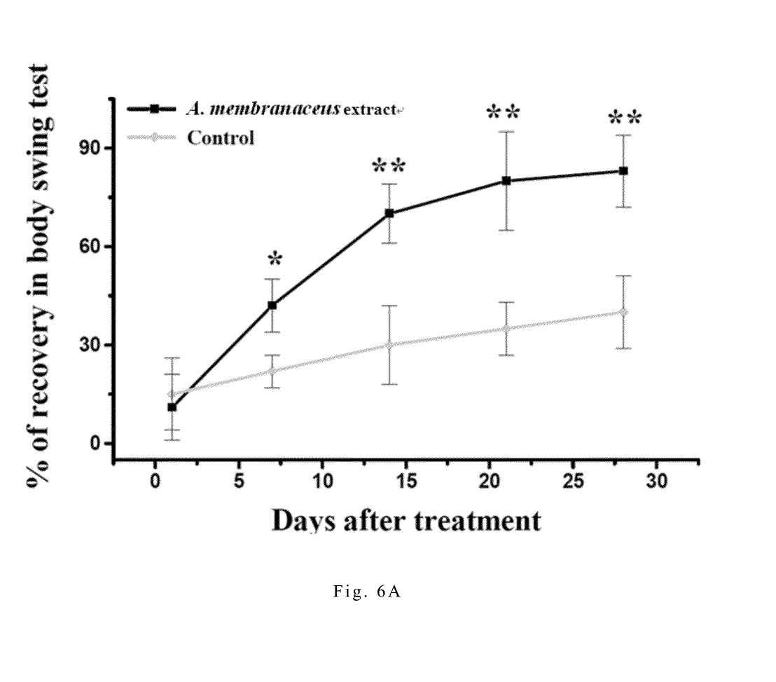

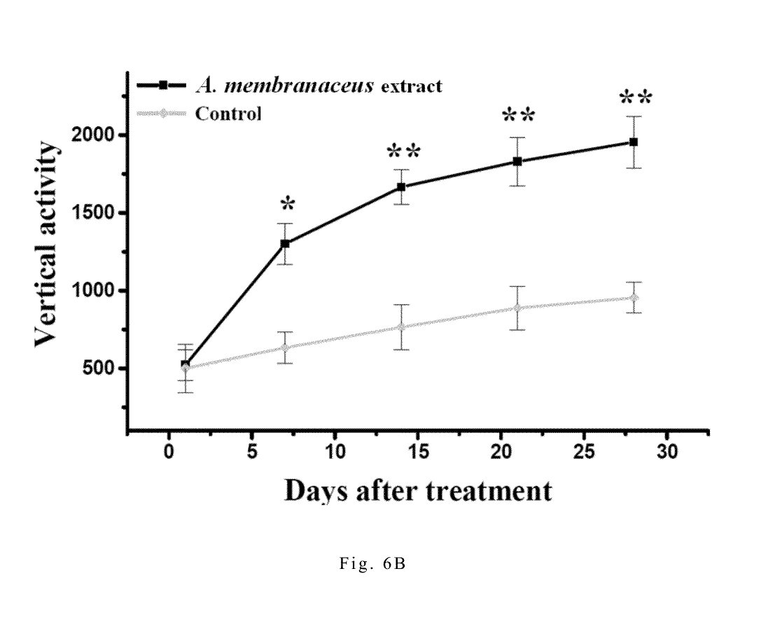

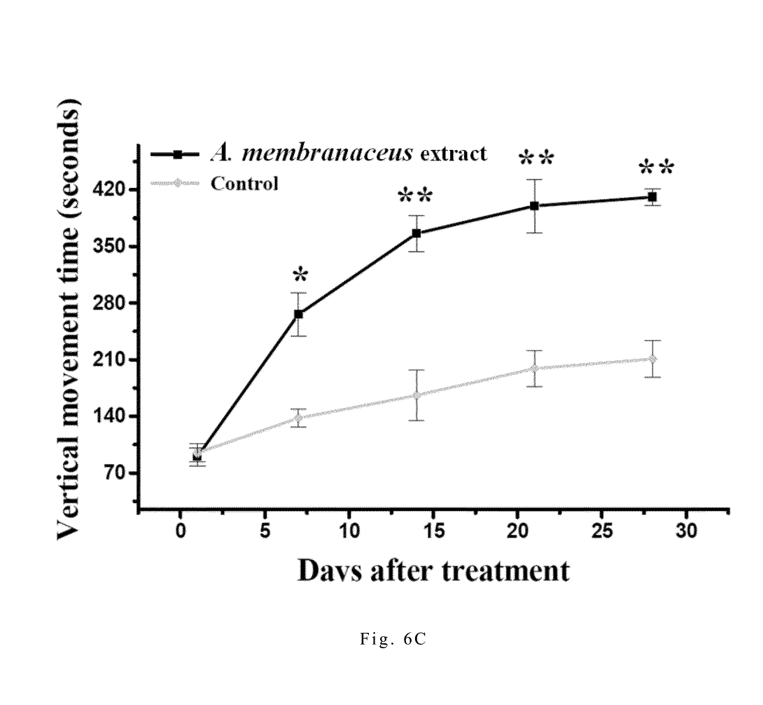

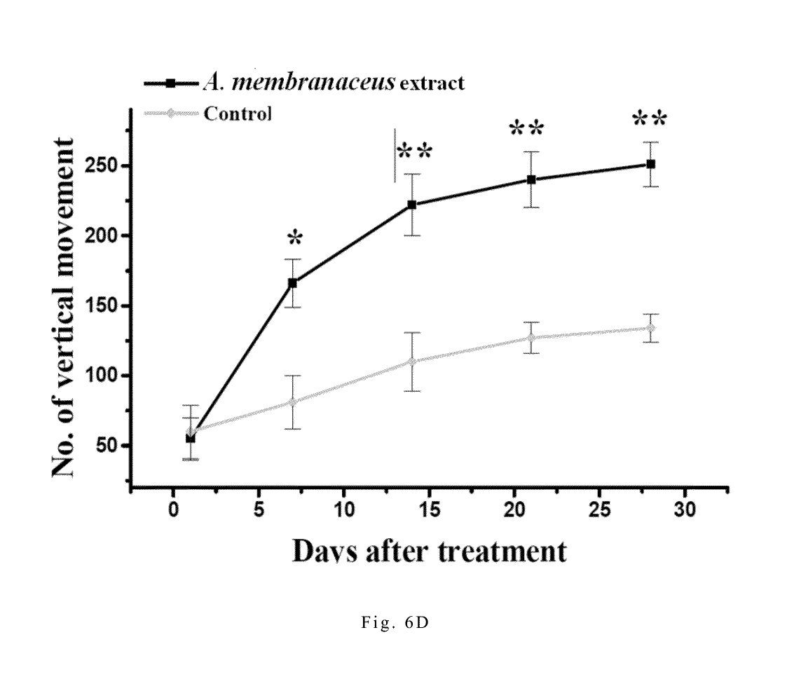

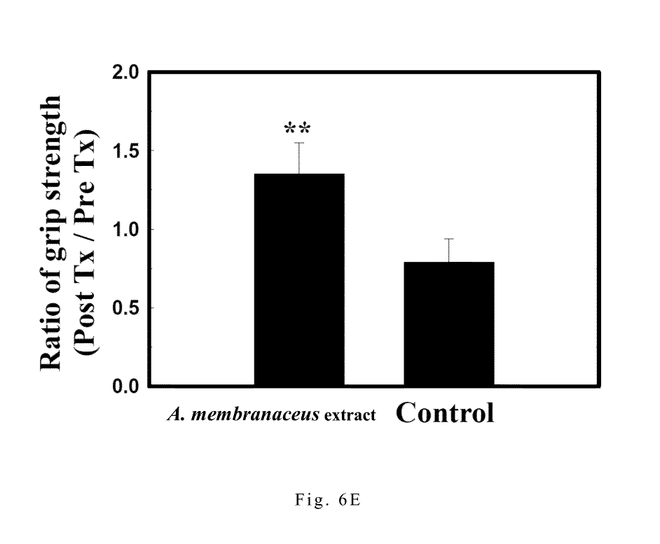

According to this mechanism, we proposed the supplement of exogenous the Astragalus membranaceus extract as therapeutic approach may promote the functional recovery after stroke. To select the most effective treatment dosage of the Astragalus membranaceus extract, TTC staining was measured in three rat groups treated with 1, 10, 25, 50 mg/kg of the Astragalus membranaceus extract. The infarct volume of the rats given 50 mg/kg the Astragalus membranaceus extract was much smaller than that in the other dosage groups at 3 days after cerebral ischemia (FIG. 5A˜FIG. 5B). At 7 days after cerebral ischemia, the infarct volume and area of the largest infarcted slice as assessed by magnetic resonance imaging were significantly reduced in the Astragalus membranaceus extract treated rats (50 mg/kg the Astragalus membranaceus extract) as compared with vehicle control (FIG. 5C˜FIG. 5E). Next, body asymmetry, locomotor activity tests, and grip strength measurement were used to assess neurological deficit recovery in the Astragalus membranaceus extract-treated, or saline control. The Astragalus membranaceus extract-treated rats showed better recovery in body swing tests than did rats treated saline control (FIG. 6A). Locomotor activities were substantially better after cerebral ischemia in rats receiving the Astragalus membranaceus extract as compared with control groups (FIG. 6B˜FIG. 6D). In addition, comparison of forelimb grip strength before and 28 days after ischemia showed that the Astragalus membranaceus extract treated group had a much better grip strength ratio than did control groups (FIG. 6E).

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following

Most important of all is the finding that Astragalus membranaceus extract is effective in reducing brain injury and improving functional outcomes in animal and human studies. In summary, Astragalus membranaceus extract is a superb candidate for preventing and treating brain injury.

1. A composition for use in the treatment of brain injury comprising an effective amount of Astragalus membranaceus extract and pharmaceutically acceptable carrier, excipients or salts.

2. The composition for use according to claim 1, wherein the Astragalus membranaceus extract comprise:
60%-120% totally polysaccharides;
α-1,4 glycosidic bond linkage;
α-1,6 glycosidic bond linkage;
bond linkage of arabinose at terminal carbon, C3 and C5;
bond linkage of glycan at terminal carbon, C4 and C6; bond linkage of galactose at terminal carbon, C3 and C6;
bond linkage of galacturonic acid at C4; and
bond linkage of rhamnose at C2.

3. The composition for use according to claim 1, wherein the brain injury include the traumatic brain injury (TBI) and the acquired brain injury (ABI).

4. The composition for use according to claim 1, wherein the treatment situation include prevention, cure or prognosis.

5. The composition for use according to claim 1, wherein the effective amount of Astragalus membranaceus extract is between 125 mg˜2000 mg/day.

6. The composition for use according to claim 1, wherein the effective amount of Astragalus membranaceus extract can cure the sequela of the brain injury and increase the patients recovery.

7. A composition for use in the treatment of the cerebral stokes comprising an effective amount of Astragalus membranaceus extract and pharmaceutically acceptable carrier, excipients or salts.

8. The composition for use according to claim 7, wherein the Astragalus membranaceus extract comprise:
60%-120% totally polysaccharides;
α-1,4 glycosidic bond linkage;
α-1,6 glycosidic bond linkage;
bond linkage of arabinose at terminal carbon, C3 and C5;
bond linkage of glycan at terminal carbon, C4 and C6; bond linkage of galactose at terminal carbon, C3 and C6;
bond linkage of galacturonic acid at C4; and
bond linkage of rhamnose at C2.

9. The composition for use according to claim 7, wherein the cerebral stroke is the cerebral ischemia stroke or the cerebral haemorrhage stroke.

10. The composition for use according to claim 7, wherein the treatment situation include prevention, cure or prognosis.

11. The composition for use according to claim 7, wherein the effective amount of Astragalus membranaceus extract is between 125 mg˜2000 mg/day.

12. The composition for use according to claim 7, wherein the effective amount of Astragalus membranaceus extract can cure the sequela of the cerebral stroke and increase the patients recover.

13. A method for preparing extract of Astragalus membranaceus comprising:
1) wash and clean the dry of Astragalus membranaceus to get a clean Astragalus membranaceus and further mechanically processed into drink chips or fine powder.
2) put the processed Astragalus membranaceus drink chips or fine powder into an extract solution to extract at temperature 80° C. to 100° C., for 2 hrs to 3 hrs to get the a first extract solution, then repeat 2 times at same temperature, for 1.5 hrs to 3 hrs.
3) concentrate all of the extract solutions, then use ethanol or lower alkanol to be precipitated and separated at low temperatures; and
4) put the selected precipitates though centrifugation, ultra filter and/or ion exchanger, following high molecular weight cut-off membrane.
